(19) United States
(12) Patent Application Publication
(10) Pub. No.: US 2023/0067950 A1
(43) Pub. Date: Mar. 2, 2023

(54) NON-CONTACT BODY TEMPERATURE SYSTEM AND METHOD

(71) Applicant: Safety Shield Products, LLC, Delray Beach, FL (US)

(72) Inventors: William Burbank, Delray Beach, FL (US); Tom Benstein, Delray Beach, FL (US)

(73) Assignee: Safety Shield Products, LLC, Delray Beach, FL (US)

(21) Appl. No.: 17/465,041

(22) Filed: Sep. 2, 2021

(51) Int. Cl. H04N 7/18; H04N 5/33; H04W 4/80; G06K 9/00; A61B 5/00

(52) U.S. Cl. CPC H04N 7/186; H04N 5/33; H04W 4/80; G06K 9/00228; A61B 5/0008

(57) ABSTRACT

A system for non-contact body temperature determination includes a temperature sensor unit having a temperature sensor configured to detect a body temperature of a human subject within a field of view of the temperature sensor. The system also includes a computing device having a user interface and configured to wirelessly communicate with the temperature sensor unit, to receive body temperature information from the temperature sensor unit, and to display the body temperature information to a user. In some embodiments, the system includes a video doorbell assembly having a doorbell button, a microphone, a speaker, and an optical camera. Also disclosed is a method of determining a body temperature of a visitor at a building entrance equipped with a video doorbell.

### TECHNICAL FIELD

The present disclosure relates generally to devices for detecting body temperature and more specifically to a non-contact body temperature sensor that can be used with a security system or video doorbell.

### BACKGROUND

Video doorbells enable a homeowner to view and communicate with a visitor at the door. For example, by activation via a motion sensor or when the visitor presses the doorbell, a video doorbell turns on to capture video of the area in front of the doorbell. The owner receives an alert on a mobile phone or smart display and can view video of the visitor(s) in real time, whether a salesman, burglar, package delivery person, animal, neighbor, or family member, for example. Upon viewing the alert and/or video, the homeowner can speak to the visitor, admit the visitor to the property, or ignore the alert as appropriate for a given situation. Some such video doorbells are equipped with motion detection, wireless two-way audio communication, wireless video transmitted to a phone or other device, and real-time notification.

### SUMMARY

One aspect of the present disclosure is directed to a system for non-contact body temperature determination of a visitor at a building entrance equipped with a doorbell. In one embodiment, the system comprises a doorbell for a building entrance, where the doorbell is actuatable by a visitor. A temperature sensor unit is configured to be mounted adjacent the building entrance, the temperature sensor unit responsive to actuation of the doorbell, where the temperature sensor unit includes a temperature sensor configured to acquire body temperature information of a human subject within a field of view of the temperature sensor. A computing device has a user interface and is configured to wirelessly communicate with the temperature sensor unit, to receive body temperature information from the temperature sensor unit, and to display the body temperature information of the human subject to a user.

In some embodiments, the temperature sensor unit includes a first wireless transceiver; the doorbell is part of a doorbell assembly including a doorbell button, a microphone, a speaker, an optical camera, and a second wireless transceiver; and the computing device is further configured to display images captured by the optical camera. In one such embodiment, the system includes a network hub configured to enable wireless communication between any one or more of the temperature sensor unit, the computing device, and the doorbell assembly, where the computing device is configured to receive user input to control operation of one or both of the temperature sensor unit and the doorbell assembly. In another embodiment, the system includes a network hub configured to enable wireless communication between the temperature sensor unit and the doorbell assembly.

In another embodiment, a system for non-contact body temperature sensing comprises a video doorbell assembly including a doorbell button, an optical camera, a microphone, and a speaker; a temperature sensor unit disposed in proximity with the video doorbell assembly, the temperature sensor unit including a temperature sensor configured to capture body temperature information of a person within a field of view of the temperature sensor; and a computing device including a user interface; where the temperature sensor unit and the video doorbell assembly are configured for wireless communication with the computing device and the computing device is configured to receive the body temperature information from the temperature sensor unit, and to display the body temperature information to a user via the user interface.

In some embodiments, the system further comprises a server computer in communication with the computing device and with the temperature sensor unit, where the server computer is configured to process the body temperature information received from the temperature sensor unit and communicate processed temperature information to the computing device.

In some embodiments, the temperature sensor unit is configured to detect an audible chime of the doorbell assembly.

In some embodiments, the system further comprises a motion sensor disposed in communication with at least one of the doorbell assembly and the temperature sensor unit.

In some embodiments, the temperature sensor unit is further configured to determine ambient temperature.

In some embodiments, the system comprising an optical camera on the temperature sensor unit.

In some embodiments, the wireless communication includes at least one of a Bluetooth communications protocol and a Wi-Fi communications protocol.

Another aspect of the present disclosure is directed to a method of detecting a temperature of a visitor at a building entrance equipped with a doorbell, such as a video doorbell. In one embodiment, the method comprises providing a temperature sensor unit adjacent to the building entrance, the temperature sensor unit including a temperature sensor and a wireless communications transceiver; providing a computing device configured to wirelessly communicate with the temperature sensor unit; acquiring, by the temperature sensor unit, body temperature data of the visitor; communicating, by the temperature sensor unit, the body temperature data to the computing device via the wireless communications transceiver; and displaying, by the computing device, a body temperature of the visitor to a user.

In some embodiments, the method includes detecting, by the temperature sensor unit or the doorbell, a visitor at the building entrance.

In some embodiments, detecting the visitor at the building entrance includes the temperature sensor unit detecting an audible chime of the doorbell.

In some embodiments, the doorbell is a video doorbell and detecting the visitor at the building entrance includes the temperature sensor unit receiving a signal from the video doorbell.

In some embodiments, the method further includes the computing device notifying the user of a visitor at the building entrance; prompting the user for an input; receiving the input from the user; and in response to receiving the input, processing the body temperature data.

In some embodiments, acquiring the body temperature data of the visitor includes determining a facial region of the visitor and acquiring a thermal image including the facial region.

In some embodiments, displaying the body temperature of the visitor includes displaying a thermal image.

In some embodiments, detecting the visitor is performed by a motion detector in communication with the temperature sensor unit.

In some embodiments, the doorbell is part of a video doorbell assembly comprising a doorbell button, an optical camera, a microphone, and a speaker, where the method further comprises (i) the doorbell assembly communicating a press of the doorbell button to the computing device, (ii) the computing device prompting the user to acquire the body temperature data, and (iii) in response to the computing device receiving user input, the temperature sensor unit acquiring the body temperature data.

In some embodiments, the method further comprises detecting, using the optical camera, a facial region of the visitor; and determining a body temperature of the visitor based on body temperature data of the facial region.

The features and advantages described herein are not all-inclusive and, in particular, many additional features and advantages will be apparent to one of ordinary skill in the art in view of the drawings, specification, and claims. Moreover, it should be noted that the language used in the specification has been selected principally for readability and instructional purposes and not to limit the scope of the disclosed subject matter.

### BRIEF DESCRIPTION OF THE DRAWINGS

FIG. 1 illustrates a front perspective view of a video doorbell assembly and a temperature sensor unit coupled together, in accordance with an embodiment of the present disclosure.

FIG. 2 illustrates a temperature sensor unit that can be mounted to a wall or other surface adjacent an entry point, in accordance with an embodiment of the present disclosure.

FIG. 3 illustrates a front perspective view of an infrared camera and circuit board that can be used to determine temperature, in accordance with an embodiment of the present disclosure.

FIG. 4 illustrates a user interface on a computing device (e.g., a cell phone) showing relative temperature within the view and a temperature reading within a selection area, where the view has been communicated wirelessly from the temperature sensor unit to the computing device, in accordance with an embodiment of the present disclosure.

FIG. 5 illustrates a flowchart of processes in a method of detecting a visitor temperature, in accordance with an embodiment of the present disclosure.

FIG. 6A illustrates a diagram showing components of a doorbell system that includes a temperature sensor unit, a doorbell, and a computing device, in accordance with one embodiment.

FIG. 6B is a diagram showing resource allocation for processes in a method of detecting visitor temperature using the system of FIG. 6A, in accordance with an embodiment of the present disclosure.

FIG. 7A illustrates a diagram showing components of a system configured to detect the temperature of a visitor, in accordance with another embodiment.

FIG. 7B is a diagram showing resource allocation for processes in a method of detecting visitor temperature using the system of FIG. 7A, in accordance with an embodiment of the present disclosure.

FIG. 8A illustrates a diagram showing components of a system configured to detect temperature of a visitor, in accordance with yet another embodiment.

FIG. 8B is a diagram showing resource allocation for processes in a method of detecting temperature of a visitor and using the system of FIG. 8A, in accordance with an embodiment of the present disclosure.

FIG. 9 illustrates a diagram of a system that includes a communications hub, in accordance with another embodiment.

FIGS. 10A-10E illustrate examples of a user interface on a user's computing device, in accordance with some embodiments.

The figures depict various embodiments of the present disclosure for purposes of illustration only. Numerous variations, configurations, and other embodiments will be apparent from the following detailed discussion.

### DETAILED DESCRIPTION

### General Overview

Video doorbells and home security systems allow the user to identify and communicate with visitors to a door or other access point. However, these systems do not provide any information to the user about the visitor's health. In particular, it would be desirable to detect the body temperature of a visitor and to alert the homeowner or building occupant to the possibility of exposure to disease, such as COVID-19, based on the visitor's body temperature.

To address this need and others, and in accordance with an embodiment of the present disclosure, a temperature sensor system configured to detect the body temperature of a visitor and communicate that temperature information to the user, such as by displaying a value on the user's computing device, such as mobile phone, smart display, or computer. Body temperature information gives the user, such as a homeowner or dwelling occupant, peace of mind by providing much-needed body temperature information about the visitor that can be used to avoid potential exposure to COVID-19 and other illnesses. In addition to providing body temperature information of the visitor, some embodiments can also be cost effective, aesthetically appealing, and easily installed by the end user.

In one example, the system includes a temperature sensor unit, and a computing device in communication with the temperature sensor unit. When a visitor arrives at the front door or other entry point, the temperature sensor unit detects the visitor's body temperature and relays the temperature information to the user's computing device to be displayed to the user. Optionally, the system also includes a video doorbell assembly, which can include an optical camera, a microphone, and a speaker, and which is configured to communicate with the user's computing device. The temperature sensor unit can operate together with or independently of the video doorbell assembly.

In one example embodiment, the temperature sensor unit is configured to detect a visitor event, such as an audible chime resulting from the visitor pressing the doorbell button on the video doorbell assembly, or the presence of a visitor indicated by triggering a motion sensor. Upon detecting the visitor event, the system alerts the user with a notification displayed on the user's computing device. Either automatically, or in response to the user pressing a button, the temperature sensor unit detects the body temperature of the visitor and displays that information to the user on the computing device. In one embodiment, the system displays a user interface on the user's computing device for viewing real-time body temperature information of a visitor.

In some embodiments, the system can include or can operate with an existing video doorbell assembly, such as one equipped with an optical camera, microphone, and communications capabilities. The temperature sensor unit can communicate directly or indirectly with the video doorbell assembly, or the temperature sensor unit can be configured to operate independently of the video doorbell assembly.

In one example, the system uses an application programming interface (API) to communicate between the user's computing device, the video doorbell assembly, and the temperature sensor unit. For example, the system is configured to detect body temperature using the temperature sensor unit and relay temperature information to the user's computing device. The system further enables communication between the user and the visitor using the user's computing device and the video doorbell assembly's optical camera, microphone, and speaker. In one such embodiment, software of an existing video doorbell or security system can be modified to display temperature information on the user's computing device, and to utilize the communications capabilities of the video doorbell assembly software for communication between the user and the visitor. Accordingly, the user interface on the computing device can be used to communicate with the visitor using the video doorbell assembly in addition to displaying and/or processing body temperature information received from the temperature sensor unit, for example.

In another example, a doorbell system includes a temperature sensor unit, such as an infrared (IR) sensor or camera, that can be triggered by a motion sensor or some other event. After detecting a visitor event, such as a visitor pressing the doorbell button or the visitor being detected by a motion detector, the temperature sensor determines the visitor's body temperature, whether a single point reading or by acquiring a temperature map of objects in view of a thermal imaging camera, for example. The temperature data captured by the temperature sensor can be communicated to the user's computing device. In some embodiments, after receiving an alert that a visitor is at the door, the user can proceed to select a particular region or point of interest within a field of view to be analyzed for temperature information. In one example, a processor analyzes a thermal image or video to determine temperature data, such as high and low temperatures or the temperature data within a particular region in the field of view. In another example, the temperature sensor detects the temperature at a location selected by the user.

Systems and components in accordance with the present disclosure can be used at the entrance to a house, condominium, apartment, a small business, or any location where a doorbell is used, including a non-store retailing business selling goods or services outside the confines of a retail facility, a service repair center, a mail order facility, a telemarketing office, a small business with limited number of employees, and a business that operates by appointment only. Numerous variations and embodiments will be apparent in light of the present disclosure.

### Example Embodiments

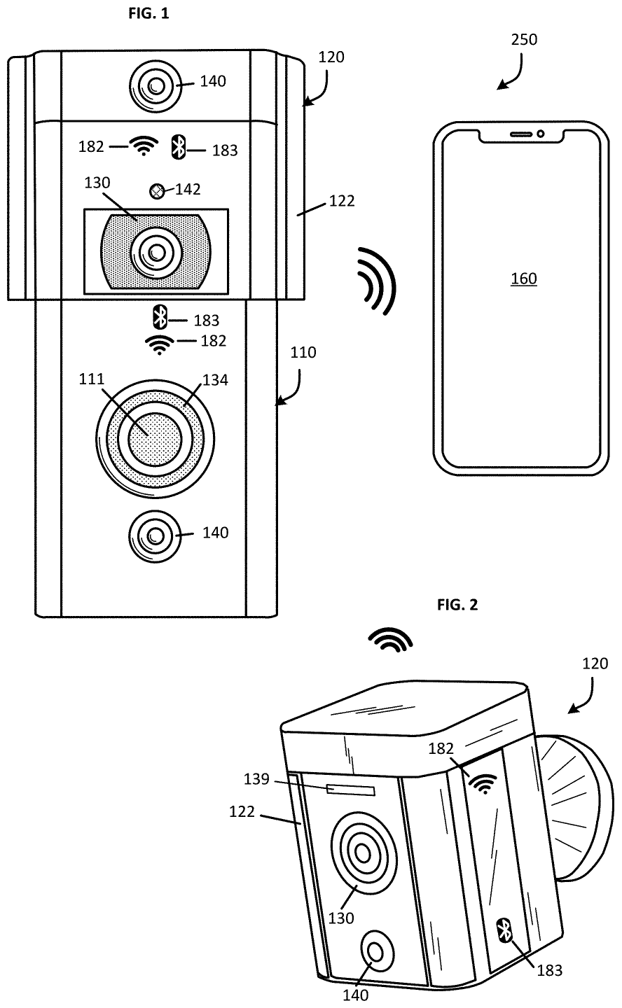

FIG. 1 illustrates a front perspective view of a doorbell assembly 110, a temperature sensor unit 120, and a computing device 160 as components of a system 250, in accordance with an embodiment of the present disclosure. The doorbell assembly 110 and temperature sensor unit 120 are part of a system 250 that also includes a computing device 160 in communication with the doorbell assembly 110 and/or the temperature sensor unit 120. The computing device 160 can be a smartphone, a tablet computer, a personal computer, a smart display, a television, a smart watch, or other wired or wireless computing device. In one example, the computing device 160 is a portable Internet-enabled electronic product that includes a display screen, speaker, microphone, and camera, and that enables the user to interact with other Internet-enabled devices, cell phones, security devices, and the like. Examples of a smart display include the electronic products sold as the Amazon Echo, Amazon Alexa, Google Home Mini.

The doorbell assembly 110 is configured as a video doorbell assembly and includes a doorbell button 111, a microphone/speaker 134, and an optical camera 140. The doorbell assembly 110 is equipped with a Wi-Fi transceiver 182 for wireless communication with the user's computing device 160. In some embodiments, the doorbell assembly 110 includes a Bluetooth transceiver 183.

In this example, the temperature sensor unit 120 is mounted to the doorbell assembly 110. For example, the housing 122 is shaped to adhere to or be otherwise fixed to the doorbell assembly 110. The temperature sensor unit 120 includes a housing 122 that retains a temperature sensor 130, such as a thermal imaging camera, and associated circuitry needed to detect a body temperature, whether at a single point or a temperature map within a field of view of the temperature sensor unit 120. One such temperature sensor 130 is an infrared camera configured to capture video and/or still images. Optionally, the temperature sensor unit 120 includes an optical camera 140 and/or an ambient temperature sensor 142. For example, the optical camera 140 can be used for facial recognition and/or to display a view of the temperature sensor to the user. The ambient temperature sensor 142 can be a thermocouple, digital thermometer, or other component configured to detect ambient temperature in the nearby region of the temperature sensor unit 120.

In some embodiments, the temperature sensor unit 120 is configured to communicate directly or indirectly with the doorbell assembly 110 via wired or wireless means. In one example, the temperature sensor unit 120 has a wired connection to the doorbell assembly 110. In another example, the temperature sensor unit 120 may include one or both of a Bluetooth transceiver 183 and a Wi-Fi transceiver 182. For example, the temperature sensor unit 120 uses the Bluetooth transceiver 183 for communication with the doorbell assembly 110 and with the user's computing device 160 at short range, such as when the user is at home. The temperature sensor unit 120 can use the Wi-Fi transceiver 182 for communicating with the user's computing device 160 when the user is away from the dwelling. For example, the temperature sensor unit 120 joins the residence's (or building's) local Wi-Fi network to communicate with the resident's computing device 160 via the network's wireless router. For example, the local Wi-Fi network can be a wireless network of a home or business and includes a wireless router. The local Wi-Fi network typically is within the building housing the entrance where entry is sought, but the system can also use a nearby wireless network in some embodiments.

In some embodiments, the temperature sensor unit 120 wirelessly communicates captured temperature information to the user's computing device 160 using cloud-based software and a wireless communications protocol, such as IEEE 802.11 for Wi-Fi communications. In some embodiments, the system 250 includes a microphone 134, a Bluetooth transceiver 183, and a power source. The power source can be a rechargeable lithium-ion battery, replaceable batteries, or wiring to a mains power supply, a solar panel, or other suitable power source. In some embodiments, the system 100 includes an optical camera 140 suited to capture video and/or still images. In some embodiments, the optical camera 140 can be used to capture images or video that are processed by the processor 148 to detect a face or facial region. In some embodiments, the system 100 includes an ambient temperature sensor 142, such as a digital thermometer or thermocouple.

System 250 includes a processor 148 configured to processes images, video, or temperature data captured by the temperature sensor 130 and/or the optical camera 140. The processor can be cloud-based software or hardware (e.g., a server computer “in the cloud”), the user's computing device, firmware and/or hardware in the temperature sensor unit 120, or a combination thereof. After capturing temperature information or detecting the visitor's body temperature, the temperature sensor unit 120 processes the raw captured data. Processing may additionally or alternately be performed by a server computer “in the cloud,” by the user's computing device 160, or a combination of such devices.

In some embodiments, the system 250 is configured to determine whether the visitor's facial area is covered by a mask or clothing, and during processing excludes covered areas of the facial region from a “hot spot” in the temperature measurement region. Determining face covering can be performed, for example, by detecting a facial region of the visitor and analyzing the temperature measurements of the facial region. If the temperature gradients within the facial region deviate more than a predetermined amount with temperature gradients of an uncovered facial region stored in the system, the system 250 identifies one or more facial coverings. For example, if a visitor's is completely covered or covered to the extent that a reliable temperature measurement cannot be obtained (e.g., visitor dons a hat, sunglasses, and mask covering the nose and lower face), the system 250 alerts the user of this condition. Optionally, the system 250 prompts the user to communicate with the visitor to remove some or all of the face covering.

FIG. 2 illustrates a temperature sensor unit 120 that can be mounted to a wall adjacent to a door or other point of entry, in accordance with an embodiment of the present disclosure. For example, the temperature sensor unit 120 can be mounted within a range of 10 meters of the door, including within 3 meters, within 1 meter, within 0.5 meter, and within 0.1 meter. In some embodiments, the temperature sensor unit 120 includes a temperature sensor 130, such as a thermal imaging camera or other suitable device for detecting the body temperature of a visitor. Similar to the embodiment shown in FIG. 1, the temperature sensor unit 120 of FIG. 2 optionally can include an optical camera 140, ambient temperature sensor 142, microphone 134, Bluetooth transceiver 183, Wi-Fi transceiver 182, and/or a motion sensor, as deemed appropriate. When mounted separately from the doorbell assembly 110, the temperature sensor unit 120 optionally can be configured to communicate directly or indirectly with the doorbell assembly 110 using wireless means (e.g., Bluetooth transceiver 183), although a wired connection can also be used. In other embodiments, the temperature sensor unit 120 is triggered by motion or sound. In some such embodiments, the temperature sensor unit 120 operates independently of the doorbell assembly 110 (if present).

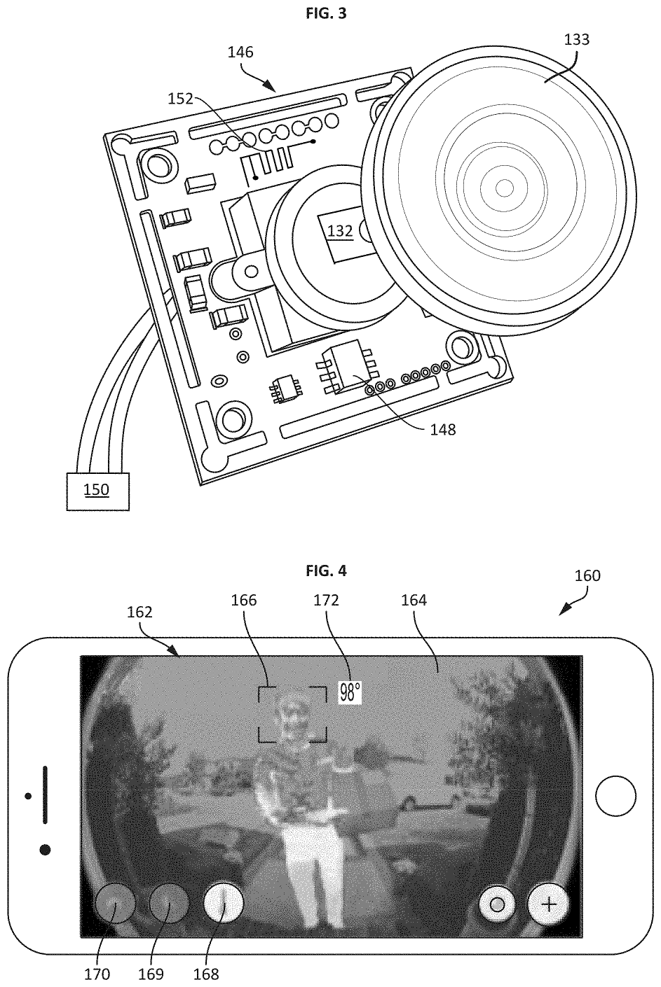

FIG. 3 illustrates a front perspective view of a circuit board 146 that includes a temperature sensor 130 configured as a thermal imaging camera, in accordance with one embodiment. In this example, the thermal imaging camera includes a thermal sensor 132 positioned behind a lens 133 and configured to receive light through the lens 133. In some embodiments, the circuit board 146 includes a processor 148 configured to control the temperature sensor 130 and to process acquired raw temperature data. The circuit board 146 can also include a transceiver 152 to communicate temperature information—a temperature value, a thermal image, or other information—to the user's computing device 160. In this example, the circuit board 146 is wired to a power supply 150, such as a battery or energized line.

In some embodiments, the temperature sensor 130 includes an integrated field-of-view Passive Infrared Sensor (PIR). For example, the temperature sensor 130 is configured to capture a temperature image within a field of view of the lens 133. Further, in some embodiments, the PIR sensor can be used with the system software to display a frame 166 on the user's computing device 160, where the frame 166 identifies a temperature measurement zone to enable the user to determine whether the visitor is appropriately positioned in the field of view and/or range of the temperature sensor 130.

In one embodiment, the temperature sensor includes a Far Infrared Sensor (FIR) to measure the ambient temperature (e.g., outdoor temperature). The system 250 can determine the ambient temperature in a periodic or continuous fashion. The time period between ambient temperature measurements can be 1 minute, 2 minutes, 5 minutes, 10 minutes, 30 minutes, or some other amount of time. For example, the processor 148 receives ambient temperature measurement data from the FIR sensor of the temperature sensor 130 every 5 minutes. Based on the ambient temperature, the processor 148 can, if needed, compensate for the influence of ambient temperature when determining a visitor's facial skin temperature in order to provide the most accurate body temperature reading. Other acceptable temperature sensors 130 include a far infrared thermal sensor array, a thermal imaging camera, a thermopile sensor, a pyrometer, and a bolometer.

In some embodiments, the system 250 is configured to recognize covered facial areas within the frame 166 and excludes covered areas from being identified as a “hot spot” on the thermal image 164 displayed on the user interface 162. In one example, the processor 148 uses an optical camera 140 to detect a face, and based on the temperature values or gradients within that facial region being outside of expected values, the processor 148 can determine whether a visitor has too much of the face covered or has particular regions of the face covered. For example, if the visitor is wearing a facemask, a hat, and/or glasses, or if the visitor's forehead is blocked from view by a face covering or hat, the system 250 can alert the user with a recommendation to ask the visitor to remove the face covering item(s) so that the system 250 can provide an accurate temperature reading.

FIG. 4 illustrates a computing device 160 (e.g., a cell phone) with a user interface 162 (e.g., a touch screen) showing a thermal image 164 captured by the temperature sensor 130, in accordance with an embodiment of the present disclosure. The thermal image 164 was received from the temperature sensor unit 120 via a wired or wireless connection to the Internet, a local area network (LAN), a wide area network (WAN), or some other communications network. In this example, the thermal image 164 is displayed with various colors each of which is indicative of a temperature range. In one example, blues and greens represent temperatures up to 75° F., yellow is indicative of temperature from 75-85° F., orange is indicative of temperature from 85-95° F., and red is indicative of temperature from 95-105° F. Other colors can also be used to indicate other temperature ranges, such as temperatures above normal body temperature. In some embodiments, the thermal image 164 is displayed in two tone or monochromatic mode, where a first color or intensity indicates a temperature equal to or above a predefined threshold (e.g., normal body temperature) and a second color or intensity indicates temperatures above the predefined threshold.

The computing device 160 includes software configured to prompt the user and to receive user input via a touch screen, keyboard, mouse, microphone, or some other input method. In one example, the user can use the touch screen of the user interface 162 to select the location and size of a region of interest enclosed by a frame 166, such as by dragging and pinching touch operations. For example, the user may move the frame 166 to any portion of the thermal image 164. After moving the frame 166, or if the computing device 160 does not receive input for a predetermined length of time, the software may automatically accept the frame's location and process (e.g., display) temperature information within the frame 166. Alternately, the computing device 160 can continuously display temperature information of the objects in the frame 166 and update the display when the user moves frame 166.

In some embodiments, the user may first move and/or size the frame 166 as needed, followed by pressing a temperature processing button 168 or the like to initiate collection and processing of temperature information within the frame 166. In this example, the frame 166 includes a view of a visitor's face. Optionally, the user interface 162 includes buttons 169, 170 (or functional equivalent) for determining the high and low temperatures within the thermal image 164 or within the frame 166, depending on how the software is configured. In some embodiments, the user interface 162 displays or otherwise communicates one or more temperatures 172 to the user, whether an average temperature, a minimum temperature, and a maximum temperature, for example.

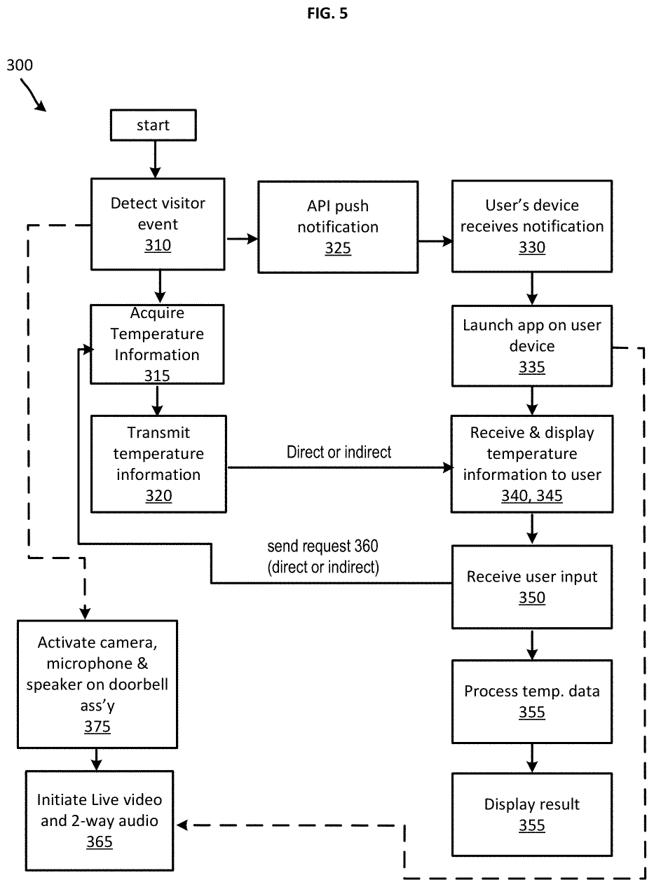

FIG. 5 illustrates a flowchart of processes in a method 300 of detecting body temperature of a visitor, in accordance with some embodiments of the present disclosure. Some embodiments of method 300 can be performed with a system 250 that includes a doorbell assembly (e.g., a video doorbell), a temperature sensor unit 120, an application programming interface (API), and a software application on the user's computing device 160, such as a personal computer, a laptop computer, a tablet computer, or a mobile phone. Other configurations of system 250 can be used as will be apparent in light of the present disclosure.

In one example, the doorbell assembly 110 is a video doorbell assembly that includes a video camera, a doorbell button, a microphone, and a speaker. The system 250 also includes a temperature sensor unit 120 with a temperature sensor 130, such as an IR thermal imaging sensor. The temperature sensor unit 120 can be integral to the doorbell assembly 110 or can be a distinct unit. In one example, the doorbell assembly 110 produces an audible chime when a visitor presses the doorbell button. In addition, or in the alternative, pressing the doorbell button may result in a signal to the processor. For example, pressing the doorbell button causes the system to open communications software on the user's computing device to enable communication via the video camera, speaker, and/or microphone. The video doorbell assembly also includes communications hardware needed to communicate with the user via wireless communications.

In some embodiments, the temperature sensor 130 includes a thermal imaging camera. In other embodiments, the thermal imaging hardware and software are part of a temperature sensor unit 120 that is distinct from the doorbell assembly 110 and that communicates directly or indirectly with the doorbell assembly 110 via wired or wireless means, where such communication can use the doorbell assembly's communications hardware or can use its own communications hardware. In one embodiment, the temperature sensor unit 120 is separate from the doorbell assembly and operates independently from the doorbell assembly. In one such embodiment, the temperature sensor unit 120 is configured to detect the audible chime of the doorbell assembly 110. Alternately, the temperature sensor unit 120 can be activated by a motion sensor or audio sensor that is part of the temperature sensor unit 120, for example.

Method 300 begins with detecting 310 a visitor event. For example, the visitor event is the presence of a visitor to an entry door of a residence or other access point. Detecting 310 the visitor event can be performed by a doorbell assembly and/or a temperature sensor unit, depending on the system configuration. Detecting the visitor event can be performed using a motion sensor, a proximity sensor, a microphone (e.g., to detect sound), the doorbell assembly receiving user input (e.g., a doorbell button touch), a change in a thermal data captured by thermal imaging camera, or some other means of the doorbell assembly or temperature sensor unit detecting a visitor to the door.

In one example, detecting 310 the visitor event includes detecting an audible doorbell chime or knocking on the door. In one such embodiment, a microphone and signal processor of the temperature sensor unit can be used to detect an audible doorbell chime or knocking, where the signal processor “looks” for an audio signal having certain characteristics, such as minimum signal strength and a particular audio wave shape consistent with a chime or knocking.

Upon detecting 310 a visitor event, method 300 continues with turning on the temperature sensor unit (if not already on) and acquiring 315 temperature information. Acquiring 315 temperature information can be performed with a temperature sensor, such as an infrared camera or other thermal imaging device. The temperature data typically includes a skin temperature of the visitor's facial region, such as the forehead or tear duct. However, the temperature information is not limited to skin temperature and can additionally or alternately include any one or more of an ambient temperature, a point temperature, light energy at one or more wavelengths, an emissivity value, a thermal image, a thermal video, a temperature gradient, a high temperature, a low temperature, and a reference temperature.

Method 300 continues with sending 320 the temperature information to the user's computing device. In some embodiments, sending 320 the temperature information utilizes an application programming interface (API) or hub to relay the information. When the configuration permits, temperature information can be sent directly to the user's computing device via a Bluetooth radio or Wi-Fi radio. In some embodiments, system 250 utilizes the temperature sensor unit, a server computer, the user's computing device, or a combination of devices to convert temperature information acquired by the temperature sensor unit to a body temperature value to be displayed to the user.

Detecting 310 a visitor event also results in the temperature sensor unit pushing 325 a notification to the user's computing device via an API. When the user's computing device receives 330 the notification from the API and/or the user acknowledges the notification, the computing device launches 335 the software application that includes a user interface (if not already running). With the software application operating, the user's computing device receives 340 the temperature information acquired by the temperature sensor unit and, after any needed processing, the computing device displays 345 the temperature information to the user via the user interface. The displayed temperature information can include one or more of a temperature map, one or more temperature values, and a status notification (e.g., a warning of high body temperature or an indication of normal temperature), for example.

Optionally, the user's computing device receives 350 user input, such as a button press or touch-screen selection. In one example, the user input can include selecting a region of interest by touching the screen, touching the screen to drag a selection box to a desired region of the display, sizing a selection field using a two-finger “pinch” technique, selecting from a list of options, speaking a selection, entering a selection using a keyboard or mouse, or other input to the computing device. In response to the received user input, the computing device processes 355 data received in step 340 and displays 355 the result to the user. Optionally, additional temperature data can be acquired by the user's computing device sending 360 a request to the temperature sensor unit (e.g., via the API) to request additional temperature data. Such additional data can be communicated to the user's computing device and displayed to the user as discussed above with processes 315, 320, 340, and 345.

In some embodiments, method 300 includes initiating 365 live video and/or 2-way audio communication with the visitor via the doorbell assembly (e.g., a video doorbell assembly) or other system component. In some embodiments, initiating 365 communication includes relaying information directly or via a hub or API to activate 375 an optical camera and microphone. In other embodiments, the doorbell assembly activates 375 the microphone and speaker as a result of detecting 310 the visitor event.

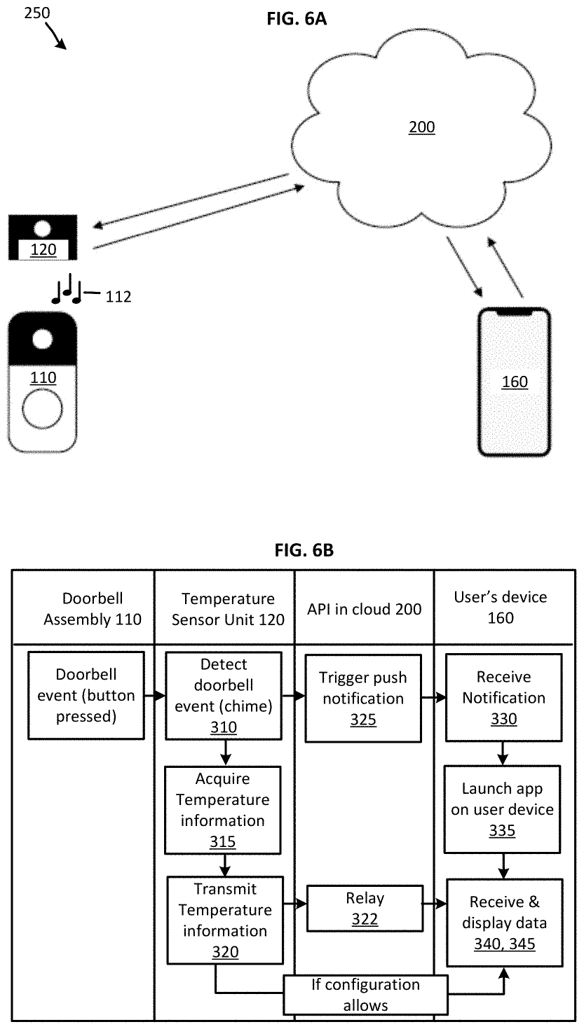

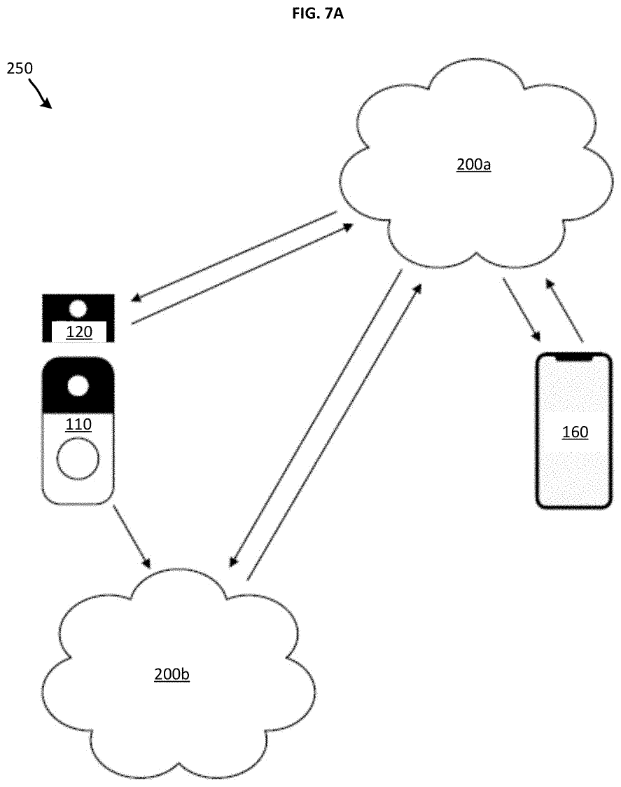

FIG. 6A illustrates a diagram showing components of a system 250 useful for performing method 300 discussed above, in accordance with one embodiment. FIG. 6B is a diagram showing processes of method 300 allocated to a particular component of the system 250. In FIG. 6B, processes are arranged in columns with the component listed in the cell above each column. Note that this particular allocation of processes and components shown in FIG. 6B is not required in all cases and some processes can be performed by a different component, depending on the particular arrangement and configuration of system components. In this example, the system 250 includes a doorbell assembly 110, a temperature sensor unit 120, and system software configured with an application programming interface (API) 200 in the “cloud” and a software application (“app”) on the user's computing device 160. The temperature sensor unit 120 is configured to detect an audible chime 112 from the doorbell assembly 110. Upon detecting 310 the chime 112 (or other visitor event), the temperature sensor unit 120 communicates with the user's computing device 160 via software and API 200. One advantage of a system as illustrated in FIG. 6A is that the temperature sensor unit 120 can be a separate component that is used with an existing doorbell assembly 110 and operates independently of the doorbell assembly 110. Accordingly, in some embodiments, the doorbell assembly 110 can be considered optional, such as when the temperature sensor unit 120 includes a motion sensor or other device to detect a visitor (a “doorbell event”), or when the temperature sensor unit 120 detects the audible chime of a traditional doorbell or a knocking sound.

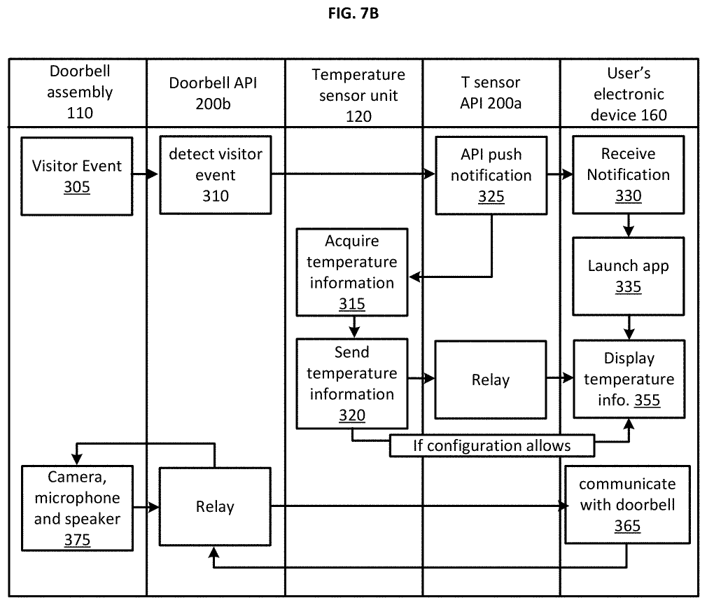

FIG. 7A illustrates a diagram showing components of a system 250 that can be used to perform method 300 discussed above, in accordance with another embodiment. FIG. 7B is a diagram showing processes of method 300 allocated to a particular component of the system 250 by column arrangement with the component listed in the cell above each column. In this example, the system 250 includes a doorbell assembly 110, a temperature sensor unit 120, and system software configured with a temperature sensor unit application programming interface (API) 200a in the cloud, system software with a doorbell application programming interface (API) 200b in the cloud, and a software application (“app”) on the user's computing device 160. The temperature sensor unit 120 is configured to communicate directly or indirectly with the doorbell assembly 110, such as a wired connection or via a wireless connection via Bluetooth, Wi-Fi, or other radio frequency communications protocol. The temperature sensor unit 120 communicates wirelessly with the user's computing device 160 via software and temperature sensor unit API 200a. The doorbell assembly 110 communicates with the computing device 160 using the doorbell API 200b. Using the doorbell API 200b and/or the temperature sensor unit API 200a, the temperature sensor unit 120, doorbell assembly 110, and user's computing device 160 communicate. Note that this particular allocation of processes and components is not required in all cases and some processes can be performed by a different component, depending on the particular arrangement and configuration of system components.

In the system 250 depicted in FIGS. 7A-7B, the temperature sensor unit API 200a can be an “unofficial” API that works with the doorbell API 200b, or an API that is certified to work with the doorbell API 200b. In embodiments where the temperature sensor unit API 200a is certified to work with the doorbell API 200b, doorbell software on the user's computing device 160 can be upgraded or modified to communicate with the temperature sensor unit 120 and display temperature information, for example. For example, the doorbell software is modified to include a user interface with a temperature display and user input to control the temperature sensor unit 120. In other embodiments, the temperature sensor unit 120 can be integral to the doorbell assembly 110 or can be a hardware add-on that is designed to operate with the doorbell assembly 110.

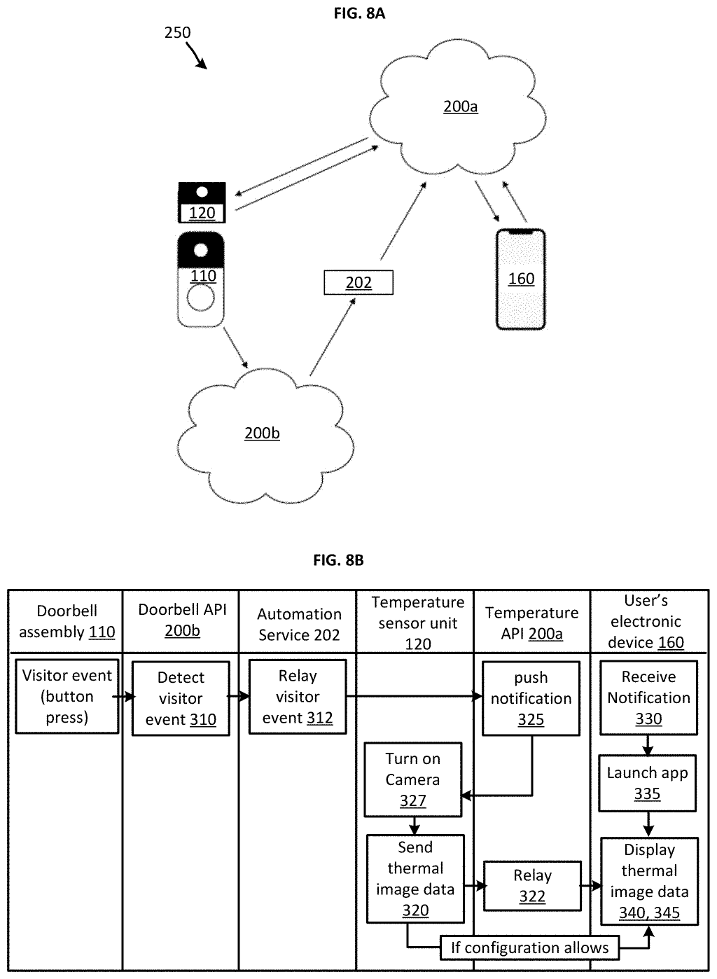

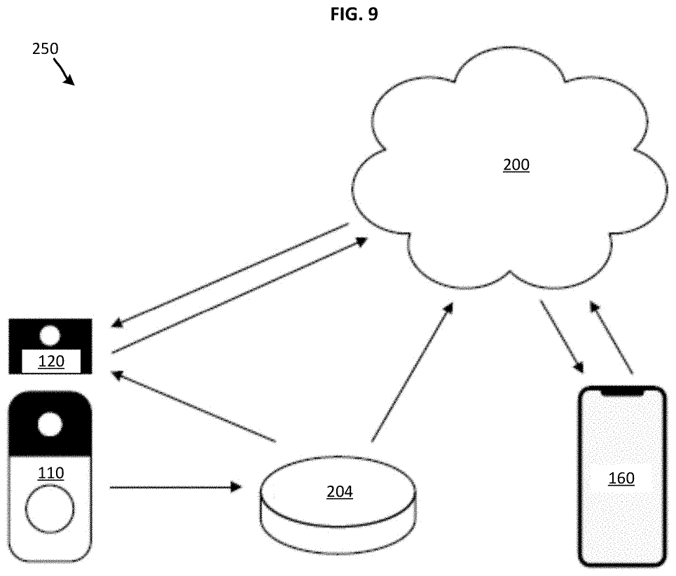

FIG. 8A illustrates a diagram showing components of a system 250 that can be used to perform method 300 discussed above, in accordance with yet another embodiment. FIG. 8B is a diagram showing processes of method 300 allocated to a particular component of the system 250 as indicated by aligning the process(es) in a column with the component listed in the cell above each column. In this example, the system 250 includes a doorbell assembly 110, a temperature sensor unit 120, system software configured with an temperature sensor unit application programming interface (API) 200a in the cloud, system software with a doorbell application programming interface (API) 200b in the cloud, a software application (“app”) on the user's computing device 160, and automation software 202. In some embodiments, the automation software 202 enables a user to program a response to events, such as communication between components in system 250 or a response to user input. One such automation software 202 is sold by If This Then That, Inc.

FIG. 9 illustrates system 250, in accordance with another embodiment. In this example, system 250 includes a network device 204 providing network communications functionality. In some embodiments, the network device 204 uses a wireless communications protocol and is configured for home automation. One such network device 204 is known as a Z-Wave Hub and utilizes a mesh network with low-energy radio waves to communicate from device to device. Such network device 204 enables wireless control of the doorbell assembly 110 and/or the temperature sensor unit 120. For example, the network device 204 functions as a hub connected to the doorbell assembly 110, the temperature sensor unit 120, and temperature sensor unit API 202a. The network device 204 provides multiple paths for communication from the doorbell assembly 110 to the user's computing device 160, namely, through the temperature sensor unit 120 and temperature sensor unit API 202a, or directly via the temperature sensor unit API 202a.

The temperature sensor unit 120 is configured to communicate with the doorbell assembly 110 using a wired connection or via a wireless connection, such as Bluetooth communication, Wi-Fi communication, or other radio frequency communications protocol. The network hub 204 receives communications from the doorbell assembly 110 and forwards those communications to an API 200 or to the temperature sensor unit 120. Accordingly, the doorbell assembly 110 can communicate with the computing device 160 via the network device and API 200. The temperature sensor unit 120 communicates with the user's computing device 160 via the API 200. Although some arrows in FIG. 9 indicate that communication may be one-directional, this is not required. In some embodiments, for example, the network hub 204 communicates data from the temperature sensor unit 120 to the doorbell assembly 110, such as to enable communication with the visitor via the doorbell assembly 110 in embodiments where the temperature sensor unit 120 operates independently of the doorbell assembly 110.

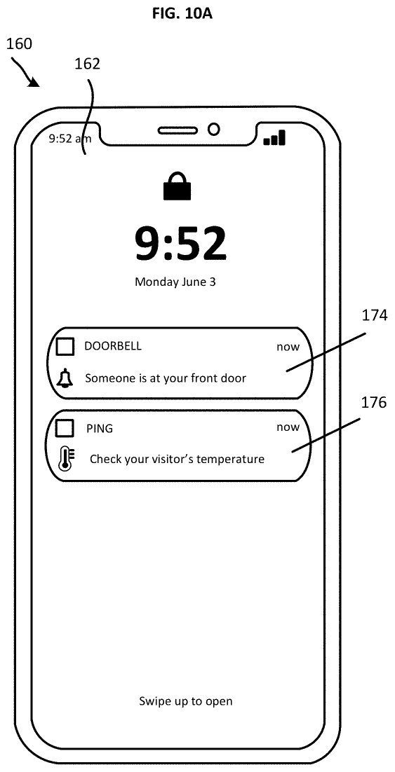

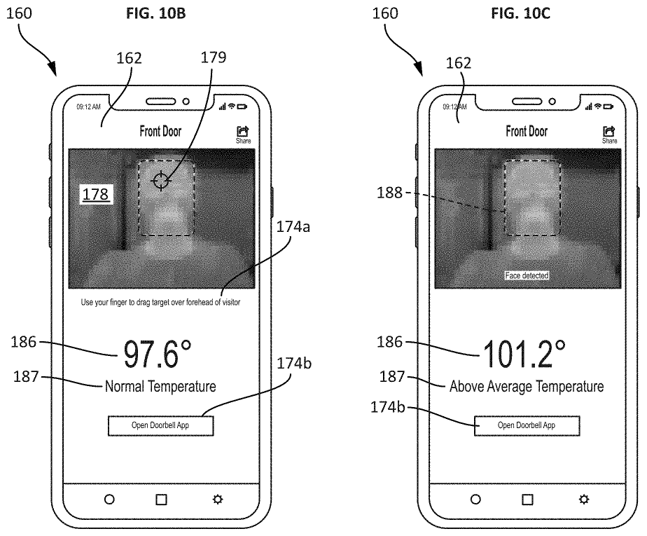

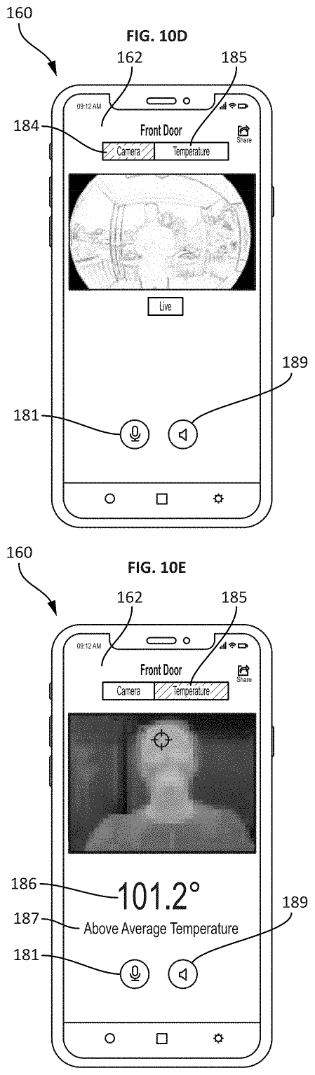

Referring now to FIGS. 10A-10E, a user interface 162 on the user's computing device 160 is illustrated at various stages of method 300, in accordance with some embodiments. In FIG. 10A, the computing device 160 displays an alert 174 indicating to the user that a visitor is at the door. Below the alert 174 is a prompt 176 to check the visitor's temperature. When the user presses the prompt 176, the system activates temperature sensing to determine a body temperature of the visitor.

FIGS. 10B and 10C show an example of the user interface 162 in an embodiment where the temperature sensor unit 120 does not communicate with the doorbell assembly 110. For example, access to an API is not available and the user must open the doorbell software application to enable communication with the visitor. In this example, the user interface 162 displays a color-coded temperature map 178 using software of the temperature sensor unit 120. The user interface 162 provides the option for the user to open the doorbell software application to initiate communication with the visitor using the doorbell assembly's 110 optical camera 140 and microphone 134.

In each of these examples, the user interface 162 displays a temperature map 178, a position selector 179 or selection box 188, and a display of the temperature 186. In the example of FIG. 10B, the displayed temperature 186 corresponds to the location where the position selector 179 is located. The user interface 162 also displays a first prompt 174a informing the user to use a finger to drag the position selector 179 to the desired area (e.g., forehead of the visitor). In FIG. 10C, the displayed temperature 186 is a high temperature within the selection box 188. Although shown displayed in these examples, the temperature 186 value is not displayed in all embodiments.

Each user interface 162 optionally displays a notice 187 indicating the status of the temperature at the position selector 179 or within the selection box 188, such as being consistent with normal body temperature or above normal body temperature. A second prompt 174b is a button that, when touched, will open the doorbell software to enable communication via the doorbell's video camera and/or microphone. In FIG. 10C, the temperature 186 reads 101.2°; accordingly, the notice 187 states that this in an abnormal body temperature. The user interface 162 in FIG. 10C also displays a selection box 188 indicating that a face has been detected.

The user interfaces of FIGS. 10D and 10E are examples of an embodiment in which API access is used to combine audio and video of the doorbell assembly 110 with temperature display of the temperature sensor unit 120. In FIG. 10D, the user interface 162 displays the video capture from the doorbell assembly's optical camera. This user interface 162 can also display temperature information received from the temperature sensor unit 120 by the user pressing the temperature icon 185. The user interface 162 of FIG. 10D also displays a camera icon 184 (shown active), a microphone icon 181, and a speaker icon 189. In this example, the camera icon 184 is illuminated as being active. Pressing the temperature icon 185 would switch the user interface 162 to display the temperature map, such as shown in FIG. 10E. The user can turn on or off the microphone by pressing the microphone icon 181 and turn on/off the phone's speaker by pressing the speaker icon 189. In FIG. 10E, the user has selected the temperature icon 185 on the user interface 162, which is now highlighted to indicate the temperature mode is active. The user interface 162 displays the temperature 186, notice 187, microphone icon 181, and speaker icon 189.

In use, embodiments of system 250 can be used to detect and communicate temperature information of a visitor to the user's computing device 160. The system 250 can be configured so that the temperature sensor unit 120 functions and communicates with the doorbell assembly 110, or so that the temperature sensor unit 120 operates independently of the doorbell assembly 110. For example, the temperature sensor unit 120 is disposed in proximity with the doorbell assembly 110, such as within communication range using a Bluetooth communications protocol, with a range of 20 feet, with a range of 15 feet, within a range of 10 feet, within a range of 5 feet, or within a range of 1 foot. Numerous variations and embodiments will be apparent in light of the present disclosure.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future-filed applications claiming priority to this application may claim the disclosed subject matter in a different manner and generally may include any set of one or more limitations as variously disclosed or otherwise demonstrated herein.

What is claimed is:

1. A system for non-contact body temperature determination of a visitor at a building entrance equipped with a doorbell, the system comprising:
a doorbell for a building entrance, the doorbell actuatable by a visitor;
a temperature sensor unit configured to be mounted adjacent the building entrance and configured to wirelessly transmit a signal, the temperature sensor unit responsive to actuation of the doorbell, wherein the temperature sensor unit includes a temperature sensor configured to acquire body temperature information of a human subject within a field of view of the temperature sensor; and
a computing device having a user interface, the computing device configured to wirelessly communicate with the temperature sensor unit, to receive the body temperature information from the temperature sensor unit, and to display the body temperature information of the human subject to a user.

2. The system of claim 1, wherein:
the temperature sensor unit includes a first wireless transceiver;
the doorbell is part of a doorbell assembly including a doorbell button, a microphone, a speaker, an optical camera, and a second wireless transceiver; and
the computing device is further configured to display images captured by the optical camera.

3. The system of claim 2, further comprising a network hub configured to enable wireless communication between any one or more of the temperature sensor unit, the computing device, and the doorbell assembly, wherein the computing device is configured to receive user input to control operation of one or both of the temperature sensor unit and the doorbell assembly.

4. The system of claim 2, further comprising a network hub configured to enable wireless communication between the temperature sensor unit and the doorbell assembly.

5. The system of claim 2, further comprising a server computer in communication with the computing device and with the temperature sensor unit, the server computer configured to process the body temperature information received from the temperature sensor unit and communicate processed temperature information to the computing device.

6. The system of claim 2, wherein the temperature sensor unit is configured to detect an audible chime of the doorbell assembly.

7. The system of claim 2, further comprising a motion sensor disposed in communication with at least one of the doorbell assembly and the temperature sensor unit.

8. The system of claim 2, wherein the temperature sensor unit is further configured to determine ambient temperature.

9. The system of claim 2, further comprising an optical camera on the temperature sensor unit.

10. The system of claim 2, wherein the wireless communication includes at least one of a Bluetooth communications protocol and a Wi-Fi communications protocol.

11. A method of detecting a temperature of a visitor at a building entrance equipped with a doorbell, the method comprising:
providing a temperature sensor unit adjacent to the building entrance, the temperature sensor unit including a temperature sensor and a wireless communications transceiver;
providing a computing device configured to wirelessly communicate with the temperature sensor unit;
acquiring, by the temperature sensor unit, body temperature data of the visitor;
communicating, by the temperature sensor unit, the body temperature data to the computing device via the wireless communications transceiver; and
displaying, by the computing device, a body temperature of the visitor to a user.

12. The method of claim 11, further comprising:
detecting, by the temperature sensor unit or the doorbell, a visitor at the building entrance.

13. The method of claim 12, wherein detecting the visitor at the building entrance includes the temperature sensor unit detecting an audible chime of the doorbell.

14. The method of claim 12, wherein the doorbell is a video doorbell and detecting the visitor at the building entrance includes receiving a signal from the video doorbell.

15. The method of claim 11, further comprising:
the computing device notifying the user of a visitor at the building entrance;
prompting the user for an input;
receiving the input from the user; and
in response to receiving the input, processing the body temperature data.

16. The method of claim 11, wherein acquiring the body temperature data of the visitor includes determining a facial region of the visitor and acquiring a thermal image including the facial region.

17. The method of claim 11, wherein displaying the body temperature of the visitor includes displaying a thermal image.

18. The method of claim 11, wherein detecting the visitor is performed by a motion detector in communication with the temperature sensor unit.

19. The method of claim 11, wherein the doorbell is part of a doorbell assembly comprising a doorbell button, an optical camera, a microphone, and a speaker, and wherein the method further comprises:
the doorbell assembly communicating a press of the doorbell button to the computing device;
the computing device prompting the user to acquire the body temperature data; and
in response to the computing device receiving user input, the temperature sensor unit acquiring the body temperature data.

20. The method of claim 19, further comprising:
detecting, using the optical camera, a facial region of the visitor; and
determining a body temperature of the visitor based on body temperature data of the facial region.
